(19) United States
(12) Patent Application Publication
(10) Pub. No.: US 2016/0309774 A1
(43) Pub. Date: Oct. 27, 2016

(54) TERPENE CARRIER

(71) Applicant: Michael D. WAND, Boulder, CO (US)

(72) Inventor: Michael D. WAND, Boulder, CO (US)

(21) Appl. No.: 15/139,660

(22) Filed: Apr. 27, 2016

Related U.S. Application Data
(60) Provisional application No. 62/153,374, filed on Apr. 27, 2015.

(51) Int. Cl. A24B 15/16; A61K 47/10; A61K 31/455; A61K 47/06; A61K 31/352; A61K 31/05

(52) U.S. Cl. CPC A24B 15/16; A61K 31/352; A61K 31/05; A61K 31/455; A61K 47/06; A61K 47/10

(57) ABSTRACT

Delivery of active ingredients by administration to an individual a vapor, aerosol, or liposomal suspension. Certain terpene solutions are employed for delivery.

### CROSS-REFERENCE TO RELATED APPLICATIONS

This application claims the benefit of U.S. provisional application 62/153,374, filed Apr. 27, 2015 which is incorporated by reference herein in its entirety.

### BACKGROUND OF THE INVENTION

This invention relates generally to the field of delivery of active ingredients to individuals, desirous of or in need of receiving such active ingredient, particularly by administration of a vapor, aerosol or a liposomal suspension to said individual. Certain terpene solutions are employed for delivery.

The terpenes and mixtures thereof of the invention are superior carriers for delivery of lipophilic active ingredients and particularly for cannabinoid active ingredients. Examples of substances that can be usefully delivered via vaporization or liposomal suspension are (tetrahydrocannabinol (THC), cannabidiol (CBD), cannabichromene (CBC), and cannabigerol (CBG). In addition, the terpenes and mixtures thereof of this invention can also be employed for delivery of nicotine, pharmaceutical compounds, vitamins, and other neutriceutical compounds.

Carriers currently used for such applications, such as propylene glycol/glycerin (PPG/G), can be toxic and inefficient for delivery, particularly of lipophilic active ingredient.

The present invention provides carriers that are less toxic and which provide more efficient delivery.

The following references may be considered relevant to this disclosure. Published patent application WO2014/100231 is incorporated by reference herein in its entirety with respect to compositions therein containing terpenes. Such disclosure can be employed to exclude certain compositions from the claims herein. Published patent application WO2006/017892 is incorporated by reference in its entirety herein particularly with respect to pharmaceutical formulations therein. Such disclosure can be employed to exclude certain compositions and methods from the claims herein. U.S. Pat. No. 5,456,247 is incorporated by reference herein in its entirety for descriptions of drug delivery employing a vaporization vehicle. Such disclosure can be employed to exclude certain compositions and methods from the claims herein. U.S. published application 2014/0166027 is incorporated by reference herein in its entirety particularly for use of electronic vapor producing devices which can be employed in the methods of this invention. Such disclosure can be employed to exclude certain compositions and methods from the claims herein.

### SUMMARY OF THE INVENTION

This invention relates generally to delivery of active ingredients to individuals, desirous of or in need of receiving such active ingredient, particularly by administration of a vapor, aerosol or a liposomal suspension to said individual. Administration can, for example, be by vaporization of a solution or suspension containing the active ingredient and subsequent inhalation of the vapor or aerosol generated, particularly where the active ingredient is substantially dissolved in the solution.

The invention additionally relates to certain terpene solutions containing certain active ingredient(s), to liposomal compositions containing certain terpenes and active ingredient(s) and to delivery formulation and pharmaceutical formulations which comprise such solutions and liposomal compositions. In addition, the invention relates to methods of delivery of one or more active ingredients employing the solutions, liposomal compositions and formulations of this invention. Terpenes of the invention are naturally-occurring terpenes that are currently used in the food and aromatherapy industries.

Examples of substances that can be usefully delivered via vaporization or liposomal suspension are (tetrahydrocannabinol (THC), cannabidiol (CBD), cannabichromene (CBC), and cannabigerol (CBG). In addition, the terpenes and mixtures thereof of this invention can also be employed for delivery of nicotine. However, many other substances, particularly active ingredients, can be delivered using the terpene carriers of this invention, including both pharmaceutical compounds, vitamins, and other neutriceutical compounds.

Mixtures containing various terpenes in concentrations of greater than 10% by weight result in superior carriers for many of these substances. Certain terpenes impart specific positive attributes such as anxiolytic, and anti-inflammatory effects, therefore increasing the mixture's overall beneficial effects, in addition to providing dramatically improved efficiency of delivery to the user. Lipophilic species are much more soluble in terpene mixtures, e.g., over 10% by weight, over 15% by weight, over 20% by weight or over 25% by weight in such mixtures.

The invention provides compositions of certain terpenes and active ingredients useful for delivery to an individual in need thereof. The invention also provides a method for delivery of one or more active ingredient comprising the steps of dissolving the one or more active ingredients in a mixture of terpenes to form a delivery composition and vaporizing the vaporizing composition. In a specific embodiment, the one or more active ingredient is one or more isolated cannabinoid receptor modulator. In another embodiment, the one or more active ingredient is nicotine. In an embodiment, the method for delivery of one or more active ingredient comprises spraying the delivery composition in contact with skin or mucosal tissue for delivery. In an embodiment, the method of delivery of one or more active ingredients comprises forming a liposomal composition comprising the delivery composition and administering the liposomal composition.

Other details of the invention are found in the following detailed description and non-limiting examples.

### BRIEF DESCRIPTION OF THE DRAWINGS

Not Applicable

### DETAILED DESCRIPTION OF THE INVENTION

This invention relates generally to delivery of active ingredients to individuals, desirous of or in need of receiving such active ingredient, particularly by administration of a vapor, aerosol or a liposomal suspension to said individual. Administration can, for example, be by vaporization of a solution or suspension containing the active ingredient and subsequent inhalation of the vapor or aerosol generated, particularly where the active ingredient is substantially dissolved in the solution. Administration can, for example, be by administration of a spray administered orally, nasally or via mucosal tissue more generally. Administration can also be by topical application to the skin employing solutions and liposomal composition herein. The invention additionally relates to certain terpene solutions containing certain active ingredient(s), to liposomal compositions containing certain terpenes and active ingredient(s) and to delivery formulation and pharmaceutical formulations which comprise such solutions and liposomal compositions. In addition, the invention relates to methods of delivery of one or more active ingredients employing the solutions, liposomal compositions and formulations of this invention.

More specifically, the invention relates to the use of certain terpenes and mixture of certain terpenes as carriers for certain active ingredients. In particularly, the terpene carriers of this invention are useful for active ingredients or combinations of active ingredients where at least one active ingredient is lipophilic. In particularly, the terpene carriers of this invention are useful for active ingredients or combinations of active ingredients where at least one active ingredient has log P of 5 or more. In more specific embodiments, the terpene carriers of this invention are useful for active ingredients or combinations of active ingredients where at least one active ingredient has log P of 5.8 or more. In more specific embodiments, the terpene carriers of this invention are useful for active ingredients or combinations of active ingredients where at least one active ingredient has log P of 6 or more. In more specific embodiments, the terpene carriers of this invention are useful for active ingredients or combinations of active ingredients where at least one active ingredient has log P of 5-7.5. In more specific embodiments, the terpene carriers of this invention are useful for delivery of cannabinoid active ingredients or combinations of such active ingredients where at least one active ingredient has log P of 5 or higher.

Terpenes of the invention are naturally-occurring terpenes that are currently used in the food and aromatherapy industries. More specifically, terpenes include those with 10, 15, 20 or 25 carbon atoms. In specific embodiments, terpene carriers of the invention comprise at least one C10 terpene (i.e., terpene having 10 carbon atoms) or a combination of two C10 terpenes. Certain mixtures of terpenes that contain higher concentrations of individual terpenes than those typically found in nature (in plants terpenes are present on the order of 0.1% up to 5% by weight), provide an excellent carrier for active ingredients, particularly for the purposes of vaporization or liposomal suspension for substances that can be delivered using these methods. As noted above, the terpenes and mixtures thereof of the invention are particularly useful for delivery of lipophilic active ingredients and particularly for cannabinoid active ingredients. Examples of substances that can be usefully delivered via vaporization or liposomal suspension are (tetrahydrocannabinol (THC), cannabidiol (CBD), cannabichromene (CBC), and cannabigerol (CBG). In addition, the terpenes and mixtures thereof of this invention can also be employed for delivery of nicotine. However, many other substances, particularly active ingredients, can be delivered using the terpene carriers of this invention, including both pharmaceutical compounds, vitamins, and other neutriceutical compounds. Mixtures containing various terpenes in concentrations of greater than 10% by weight result in superior carriers for many of these substances. Mixtures comprising such high concentrations of terpenes are superior to the substances typically used for these purposes today, such as propylene glycol/glycerin (PPG/G), and in addition to being a much less toxic alternative to the currently used PPG/G, certain terpenes impart specific positive attributes such as anxiolytic, and anti-inflammatory effects, therefore increasing the mixture's overall beneficial effects, as well as decreasing the detrimental effects of the mixture when compared to the use of PPG/G as carrier. Additionally, because of the lipophilic character of the terpene mixture, much higher concentrations of the desired substances e.g., active ingredients, such as CBD, CBC, CBG, and THC, can be dissolved in the terpene carrier resulting in dramatically improved efficiency of delivery to the user. For example, PPG/G mixtures are limited to carrying less than 10% by weight of a lipophilic active ingredient such as CBD, CBC, CBG, and THC, compared to terpene carriers, where these lipophilic species are much more soluble, e.g., over 10% by weight, over 15% by weight, over 20% by weight or over 25% by weight.

In specific embodiments, the invention provides certain terpene solutions useful for delivery of cannabinoids, particularly CBD, CBC, CBG, and THC. Such solution can be employed for vaporization delivery, spray delivery or delivery in liposomal compositions. The invention further provides methods for treatment employing such solutions and liposomal compositions. Cannabis has been used for centuries and has been shown to be effective a treatment in many disorders. However, one undesirable side effect is anxiety and/or paranoia induction by overuse of the Cannabis product. Careful selection of the right strain of cannabis can minimize these effects if the strain possesses a high concentration of CBD. In specific embodiments, combinations of one or more of CBD, CBG and CBC with specific terpenes known to lower anxiety state can be employed to reduce anxiety. Selected high concentrations of CBD and specific terpenes result in dramatically reduced or complete elimination of the anxiety state.

Terpenes are found across the plant kingdom, including in Cannabis. They show beneficial and medicinal characteristics making them excellent carriers for other active ingredients. They are particularly useful as carriers for lipophilic cannabinoids because such lipophilic species have enhanced solubility in such terpenes. Although terpenes have been used in low concentrations, generally as flavoring agents, the present work demonstrates that at least at concentrations above 10%, they can act as effective carriers for other agents. In particular, delivery of cannabinoids such as CBD, CBG and CBC in the terpene carriers of the invention result in lowered anxiety states, whether resulting from the overuse of cannabis, or generated from other non-cannabis-induced states such as PTSD, or other anxiety disorders.

Mixtures that contain concentrations of CBD at least above 10% by weight are particularly effective at reducing high anxiety and panic attack-like states, compared to mixtures that contain CBD in lower concentrations of less than 5% by weight. Terpene solutions containing at least 10% by weight or more of CBD, particularly where the terpenes comprise beta-caryophyllene and/or myrcene, is especially useful for the treatment of people that have used Cannabis to an excess, resulting in either heightened anxiety, or a paranoid state, or panic-attack.

In addition, terpene solutions containing CBD are effective at modifying the high resulting from THC into a more pleasant psychological experience, often replacing the dysphoric state with a euphoric state. Thus, administration of such solutions in combination with THC can provide particular benefit.

Certain terpenes are excellent carriers for the delivery of a wide range of drugs, active ingredients, and cannabinoids, for example, via mild heating process in a vaporization vehicle or through a liposomal suspension and delivered orally through a spray pump. These mixtures are superior to the carriers currently being used in these processes today because they are non-toxic and add positive attributes (synergistic, in particular, their anxiolytic, and anti-inflammatory effects to the compounds in the carrier.

Specifically, solutions of specific terpenes containing non-psychoactive cannabinoids are effective at dramatically reducing or illuminating the presence of increased states of anxiety, paranoia or panic, as well as acting as an anti-inflammatory agent. These beneficial effects are realized regardless of the source of the anxiety, paranoia, or panic state, it does not matter whether the state is the result of a disorder, has been induced by Cannabis use, or by circumstances present in a person's life. Although any non-psychoactive cannabinoid such as CBG and CDC can be used, the present work demonstrates that CBD is a very effective cannabinoid for reducing anxiety, paranoia, and panic. When CBD is administered in inert carriers such as glycerin, propylene glycol, and mixtures thereof, without the terpenes, it does reduce anxiety, paranoia and panic, especially when used in concentrations of at least 10%, and more preferably, greater than 50%. However, this effect is significantly enhanced when an active carrier containing terpenes such as beta-caryophyllene or myrcene and are used.

Terpenes useful in the invention include terpenes having 10 carbon atoms (C10) to 25 carbon atoms (C25). Preferred terpenes useful in the invention are terpenes having 10 carbon atoms or 15 carbon atoms. Preferred terpene mixtures are those having a predominant amount, i.e., more than 50% by weight, of C10 and C15 terpenes. Additionally preferred mixtures are those having a predominant amount, i.e., more than 50% by weight, of C10 terpenes.

In specific embodiments, the terpene solutions of the invention comprise a first terpene or mixture of first terpenes, where the first terpenes are those that do not have a strong flavor or taste, such as myrcene, beta-caryophyline or nerolidol. In specific embodiments, the terpene solution consists of a first terpene or a mixture of first terpenes. The first terpene is preferably a C10 terpene or a mixture thereof. In an embodiment, the first terpene is preferably myrcene. In an embodiment, the first terpene is beta-caryophyline. In an embodiment, the first terpene is nerolidol. In specific embodiments, the first terpene is a mixture of myrcene and beta-caryophyline. In specific embodiments, the first terpene is a mixture of nerolidol and beta-caryophyline. In specific embodiments, the terpene used in the invention is myrcene, nerolidol or beta-caryophyline or combinations thereof. In a specific embodiment, a terpene mixture is used in the invention which comprise a first terpene as noted above and a second terpene where the second terpene is present in an amount of 25% by weight or less. The second terpene can be a terpene having 10, 15, 20 or 25 carbon atoms or mixtures thereof. The second terpene preferably is a C10 or a C15 terpene. The second terpene can have a strong flavor or taste. The second terpene can be a mixture of such second terpenes where the total amount of second terpenes present is 25% by weight or less. In an embodiment, the second terpene can be selected from limonene, pinene, menthol, linalool, terpineol, terpinolene or mixtures thereof. In specific embodiments, the terpene solution comprises a first terpene or mixture of first terpenes in combination with one or more second terpenes.

In specific embodiments, the terpene solution comprises myrcene, beta-caryophyline, nerolidol or mixtures thereof in optionally combination with one or more of limonene, pinene, menthol, linalool, terpineol or terpinolene, wherein the limonene, pinene, menthol, linalool, terpineol, terpinolene or mixtures thereof is present at concentrations of 10% by weight or less. In specific embodiments, the terpene solution comprises myrcene, beta-caryophyline, or mixtures thereof in optionally combination with one or more of limonene or linalool, wherein the limonene, linalool or mixtures thereof is present at concentrations of 10% by weight or less. In specific embodiments, the terpene solution of the invention comprises myrcene, and linalool where linalool is present at concentrations of 10% by weight or less. In specific embodiments, the terpene solution of the invention comprises myrcene, beta-caryophyline and linaool, where linalool is present at concentrations of 10% by weight or less. In specific embodiments, the terpene solution of the invention comprises myrcene, and limonene where limonene is present at 10% by weight or less. In specific embodiments, the terpene solution of the invention comprises myrcene, beta-caryophyline and limonene, where limonene is present at concentrations of 10% by weight or less. In additional embodiments of the forgoing, the weight ratio of myrcene to beta-caryophyllene ranges from 4:1 to 1:4. In additional embodiments of the forgoing, the weight ratio of myrcene to beta-caryophyllene ranges from 2:1 to 1:2. In additional embodiments of the forgoing, the weight ratio of myrcene to beta-caryophyllene ranges from 1:0.8 to 0.8 to 1. In a specific embodiment, the weight ratio of myrcene to beta-caryophyllene is 1:1. In a specific embodiment, the weight ratio of myrcene to beta-caryophyllene is 3:1. In a specific embodiment, the weight ratio of myrcene to beta-caryophyllene is 2:1.

In specific embodiments, the terpene solution of the invention comprises 50% by weight or more of first terpene in combination with up to 25% by weight of second terpene and 1% to 50% by weight of active ingredient. In specific embodiments, the terpene solution of the invention comprises 99%-74% by weight of first terpene in combination with from 0% to 25% by weight of second terpene. More specifically, the terpene solution of the invention comprise from 95%-69% by weight of first terpene in combination with from 0% to 25% by weight of second terpene. Yet more specifically, the terpene solution of the invention comprises 90%-64% by weight of first terpene in combination with 0% to 25% by weight of second terpene. Yet more specifically, the terpene solution of the invention comprises 85% to 59% by weight of first terpene in combination with 0% to 25% by weight of second terpene. Yet more specifically, the terpene solution of the invention comprises 80% to 54% by weight of first terpene and 0% to 25% of second terpene. Yet more specifically, the terpene solution of the invention comprises 75% to 49% by weight of first terpene and 0% to 25% of second terpene. Yet more specifically, the terpene solution of the invention comprises 70% to 44% by weight of first terpene and 0% to 25% of second terpene. Yet more specifically, the terpene solution of the invention comprises 65% to 39% by weight of first terpene and 0% to 25% of second terpene.

In specific embodiments, the terpene solution of the invention comprises 1% to 10% by weight of second terpene. In specific embodiments, the terpene solution of the invention comprises 1% to 5% by weight of second terpene. In specific embodiments, the terpene solution comprises 5% to 10% by weight of second terpene.

In specific embodiments, the terpene solution of the invention comprises greater than 5% by weight active ingredient, 0% to 15% by weight of second terpene with the remainder being first terpene. In a specific embodiment, the terpene solution of the invention comprises 10% by weight or more of active ingredient, 0% to 15% by weight second terpene with the remainder being first terpene. In a specific embodiment, the terpene solution of the invention comprises 10% by weight or more of active ingredient, 1% to 10% by weight second terpene with the remainder being first terpene. In a specific embodiment, the terpene solution of the invention comprises 10% by weight or more of active ingredient, 5% to 10% by weight second terpene with the remainder being first terpene.

In specific embodiments, terpene solutions comprise from 1% to 50% by weight active ingredients. In specific embodiments, terpene solutions comprise 5% to 25% by weight active ingredient. In specific embodiments, terpene solutions comprise 10% by weight or more of active ingredient. In specific embodiments, terpene solutions comprise 15% by weight or more of active ingredient. In specific embodiments, terpene solutions comprise 20% by weight or more of active ingredient. In specific embodiments, terpene solutions comprise 25% by weight or more of active ingredient.

In specific embodiments, the terpene solutions comprise the first terpene in combination with one or more second terpenes. More preferred first terpenes are myrcene, neurolidol and beta-caryophylene. More preferred second terpenes are limonene, pinene, menthol, myrcene, linalool, terpineol and terpinolene.

In specific embodiments, terpene mixtures of the invention include a combination of myrcene and beta-caryophyllene. In terpene solutions containing active ingredients, terpene mixtures containing a combination of myrcene and beta-caryophyllene where the weight ratio of mycrene to beta-caryophyllene ranges from 0.8:1 to 1:0.8 are more useful for providing anti-inflammatory effect. In terpene solutions containing active ingredients, terpene mixtures containing a combination of myrcene and beta-caryophyllene where the weight ratio of mycrene to beta-caryophyllene ranges from 4:1 to 2:1 are more useful for providing anxiolytic effect. In the forgoing embodiments, beta-caryophyllene can be replaced in whole or in part with alpha-caryophyllene. In the forgoing embodiments, myrcene can be replaced in whole or in part by neurolidol.

In specific embodiments, terpene mixtures of the invention include 1% to 15% by weight limonene and more preferably 1% to 5% limonene. Limonene containing terpene solutions are preferred for use to alter the experience of THC. In specific embodiments, terpene solutions of the invention include 1% to 15% by weight linalool and more preferably 1% to 5% linalool. Linalool containing terpene solutions are preferred for use in ameliorating anxiety.

In specific embodiments, terpene mixtures of the invention include 1% to 10% by weight alpha-pinene, optionally as a bronchodilator, and more preferably 1% to 5% by weight of alpha-pinene.

In specific embodiments, terpene mixtures contain minor amounts of glycerine in the range from 0.5 to 5% by weight. In specific embodiments, terpene mixtures contain minor amounts of glycerine in the range from 0.5 to 2% by weight. Glycerine addition to the terpene solution can affect the lipohilicity of the solution and can be useful for dissolution of more hydrophilic active ingredients.

In specific embodiments, terpene mixture useful in the invention include (a) first terpene which is myrcene, neurolidol or a mixture thereof, and (b) second terpene which is linalool, limonene, alpha-pinene or any mixture thereof. In more specific embodiments, the first terpene is present in the mixture in amounts ranging from 99% by weight to 75%. More specifically, the first terpene is present in the mixture of terpenes in an amount ranging from 99% to 85%. More specifically, the first terpene is present in the mixture of terpenes in an amount ranging from 99% to 90%. Preferred second terpenes are limonene, linalool, a mixture of limonene and linalool, or a mixture of limonene or linalool with pinene.

In specific embodiments herein, the terpene mixtures and terpene solutions do not contain glycerine. In specific embodiments herein, the terpene mixtures and terpene solutions do not contain ethylene glycol or propylene glycol. In specific embodiments herein, the terpene mixtures contain only 10C, 15C, 20C or 25C terpenes including oxygenated derivatives of terpene hydrocarbons. Oxygenated derivatives of terpene hydrocarbons include mono- and diols, and ketones.

In specific embodiments, the invention provides a terpene carrier composition comprising a mixture of terpenes as a carrier for the one or more active ingredient for vaporizers or liposomal transport wherein the mixture of terpenes comprises a first terpene selected from myrcene, nerolidol, beta caryophyllene or a mixture thereof and one or more second terpenes selected from the group consisting of limonene, pinene, linalool, terpineol, terpinolene, terpineol, menthol and a mixture thereof. In more specific embodiments, the composition comprises a mixture of two or more terpenes selected from the group consisting of myrcene, beta-caryophyllene, linalool, or limonene. In more specific embodiments, the composition comprises a mixture of myrcene and beta-caryophyllene and one of linalool or limonene. In more specific embodiments, at least a portion of the myrcene in the composition is replaced by neurolidol. In more specific embodiments, the composition comprises a mixture of beta-caryophyllene, one of linalool or limonene and a combination of myrcene and neurolidol.

In more specific embodiments, the amount of first terpene present ranges from 50% to 100% by weight. In more specific embodiments, the amount of first terpene present ranges from 75% to 100% by weight. In more specific embodiments, the amount of second terpene present ranges from 1% to 25% by weight. In more specific embodiments, the amount of second terpene present ranges from 1% to 15% by weight. In more specific embodiments, the amount of limonene present ranges from 1% to 15% by weight. In more specific embodiments, the amount of linalool present range from 1% to 15% by weight. In more specific embodiments, In more specific embodiments, the first terpene is a mixture of myrcene and beta-caryophyllene, where the weight ratio of myrcene to beta-caryophyllene ranges from 4:1 to 1:4. In more specific embodiments, the weight ratio of myrcene to beta-carylophyllene ranges from 1:2 to 1:4. In more specific embodiments, the weight ratio of myrcene to beta-carylophyllene ranges from 4:1 to 2:1. In more specific embodiments of the forgoing embodiments, linalool is present at 5% to 10% by weight. 15. In more specific embodiments of the forgoing embodiments, limonene is present at 5% to 10% by weight.

In more specific embodiments of the forgoing embodiments, the weight ratio of myrcene to beta-caryophyllene is 1:1. In more specific embodiments of the forgoing embodiments, the weight ration of myrcene to beta-caryophyllene is 1:3

In specific embodiments, a composition of the invention comprising terpenes as descried herein further comprises one or more isolated cannabinoid receptor modulator wherein the isolated cannabinoid receptor modulator is solubilized in the terpene mixture. In more specific embodiments, the composition comprises 10% by weight of one or more isolated cannabinoid receptor modulators. In more specific embodiments, the composition comprises 15% by weight of one or more isolated cannabinoid receptor modulators. In more specific embodiments, the composition comprises 20% by weight of one or more isolated cannabinoid receptor modulators. In more specific embodiments, the composition comprises 25% by weight of one or more isolated cannabinoid receptor modulators. In more specific embodiments, the one or more isolated cannabinoid receptor modulator is selected from THC, CBD, CBC, CBG or a mixture thereof. In more specific embodiments, the one or more isolated cannabinoid receptor modulator is selected from THC or a mixture of THC with CBD or CDG.

In specific embodiments, the composition comprises a mixture of terpenes as described in Table 1 as a carrier for vaporization or liposomal delivery. In specific embodiments, the composition comprises cannabidiol and a mixture of terpenes as described in Table 1 as a carrier for vaporization delivery. In specific embodiments, the composition comprises cannabidiol and a mixture of terpenes and an amphiphilic material to form liposomes as a carrier for liposomal delivery. In specific embodiments, the amphiphilic material is lecithin or phosphatidyl choline.

In specific embodiments, the composition comprises cannabigerol (CBG) and a mixture of terpenes as described above as a carrier for vaporization or liposomal delivery.

In specific embodiments, the composition comprises any cannabinoid or mixture of cannabinoids and a mixture of terpenes as described above as a carrier for vaporization or liposomal delivery. In specific embodiments, the cannabinoid or mixture of cannabinoids is present in the composition in an amount of 10% by weight or more.

In a specific embodiment, the composition comprises nicotine and any of the combination of terpenes described in Table 2 as a carrier for vaporization or liposomal delivery. In a specific embodiment, the composition comprises any pharmaceutical or neutraceutical agent and any of the mixtures of terpenes described in Table 1 as a carrier for vaporization or liposomal delivery.

In a specific embodiment, the method for delivery of one or more active ingredient comprising the steps of dissolving the one or more active ingredients in a mixture of terpenes, particularly any terpene mixture of Table 1 or Table 2 to form a delivery composition. The delivery composition can be vaporized, sprayed in contact with skin or mucosal tissue for delivery, or sprayed into the mouth or nose of the individual, for example. In another embodiment, a liposomal composition is formed comprising the delivery composition and administering the liposomal composition to the individual.

In a specific embodiment, a terpene mixture comprises (by weight): 40-75% myrcene, 15 to 50% beta-caryophyllene, and one or both of limonene, linalool at total weight % ranging from 1-5%, and optionally one or more of neurolidol (0.5 to 5% by weight), alpha-pinene (0.5 to 2% by weight), terpineol or terpinolene (0.5 to 3% by weight) or menthol (0.5% to 7% by weight).

The term terpene is used generally herein to refer to a class of organic compounds, derived biosynthetically from units of isoprene (C₅H₈) and to variants, particularly oxygenated derivatives thereof (often called terpenoids). Terpenes typically derive from plant materials and are found for example as components of essential oils. Terpenes include: hemiterpenes which are 5C terpene hydrocarbons, e.g. isoprene, and oxygenated derivatives thereof, such as prenol and isovaleric acid (hemiterpenoids); monoterpenes which are 10C terpene hydrocarbons and oxygenated derivatives thereof including myrcene, geraniol, limonene, terpineol, pinene (α- and β-pinene), menthol, thymol, carvacrol, camphor, borneol and eucalyptol; sesquiterpenes which are 15C terpene hydrocarbons and oxygenated derivatives thereof including humulene, beta-caryophylene, neurolidol, farnesenes, and farnesol; diterpenes which are 20 C terpene hydrocarbons and oxygenated derivatives thereof such as cafestol, kahweol, cembrene and taxadiene; sesterterpenes, which are terpene hydrocarbons having 25 carbons (25C) and oxygenated derivatives thereof such as geranylfarnesol. Terpenes most useful in the present invention are those having 10 carbon atoms or 15 carbon atoms (monoterpenes and sesquiterpenes) and oxygenated derivatives thereof. Terpene mixtures of the invention can contain small amounts, i.e., less than 2% by weight or less than 1% by weight of terpenes other than monoterpenes and sesquiterpenes and oxygenated derivatives thereof.

Myrcene (more specifically beta-myrcene) is a preferred terpene for use in the present invention. Myrcene is a remarkably safe terpene. Comparing it to nicotine, the LD50 (Lethal Dose): >5 g/kg compared to nicotine at 40-60 mg/kg. Myrcene is reported to exhibit properties as an analgesic, antibacterial, mitigation of the effects of diabetes, anti-inflammatory agent, anti-insomnia agent, anti-proliferative/anti-mutagenic agent, antipsychotic and antispasmodic. Myrcene exhibits a relatively mild flavor or fragrance.

Beta-caryophyllene is another preferred terpene for use in the invention. This is classified herein as a first terpene with weak aromatic taste. This is also a safe terpene. Comparing it to nicotine, the LD50 (Lethal Dose): >5 g/kg, compared to nicotine at 40-60 mg/kg. Beta-caryophyllene is the primary terpene that contributes to the spiciness of black pepper and also a major terpene in cloves, hops, rosemary, and cannabis. In nature, beta-caryophyllene is typically found together with small amounts of its isomers isocaryophyllene] and α-humulene or in a mixture with its oxidation product. Beta-caryophyllene is unique for being both a terpene and a “dietary cannabinoid,” a food-stuff which acts as a cannabinoid and binds to CB2 receptors. This sesquiterpene is reported to be a CB receptor ligand with a fundamentally different structure from the cannabinoids, representing a new type of CB2 receptor-selective agonist. (Gertsch et al. (2008) PNAS 105(26): 9099-9104) Beta-caryophyllene is nonphyscoactive. Beta-caryophyllene is also reported to have medicinal benefits, including anti-oxidant activity, anti-inflammatory activity, anti-cancer activity and local anesthetic effects. Beta-caryophyllene is variously reported to exhibit analgesic effect, antibacterial effect, antidepressant effect, anti-inflammatory effect, anti-proliferative effect, antioxidant effect, anxiolytic effect and neuroprotective effect. Gertsch et al (2008) is incorporated by reference herein in its entirety among others for its discussion of CB1 and CB2 receptors and the properties of beta-caryophyllene.

The low boiling point of myrcene (C10H22) allows for the creation of an excellent and low toxicity carrier, superior to propylene glycol which has been known to release formaldehyde and acrolein, which are quite toxic upon heating. Beta-caryophyllene is a CB2 agonist in the endocannabinoid system, which results in a natural anti-inflammatory response, thus protecting the body's neural system from potential damage from free radicals. This results in a carrier for CBD and other cannabinoids that is less harmful than the currently accepted vaporizer carrier standard, propylene glycol/glycerin. Additionally, the mixture of terpenes in the present invention provides the beneficial effects of the anti-inflammatory CB2 agonist effect and a more pleasant taste.

Limonene is a cyclic monoterpene that has a pronounced citrus odor and flavor, somewhat sweet yet tangy and bitter. Limonene is found in highest concentrations in the rinds of citrus fruit. A major reason for limonene's widespread use is its very low toxicity. The LD50 (Lethal Dose): LD50 for limonene is >5.5 g/kg compared to that of nicotine is 40-60 mg/kg Limonene is reported to have numerous medicinal benefits including promoting weight loss, aiding digestion, and preventing gastric distress. It has been reported to be an anti-fungal agent. Limonene also shows promise as a treatment for anxiety and depression. Most interestingly, limonene has been reported to both stimulate the immune system and be an effective treatment for cancer. Limonene has been reported to be useful as an antidepressant, antifungal, anti-inflammatory, anti-Proliferative, anxiolytic, acid reflux reducer, immunostimulant.

Alpha Pinene is the main terpene in pine trees which gives them their characteristic scent. It is in turpentine and also present in high amounts in rosemary and cannabis. Although pinene has two main isomers, alpha- and beta-pinene, alpha-pinene is the dominant one found in cannabis. Pinene forms the biosynthetic base for CB2 ligands in the endocannabinoid system. Pinene can be a bronchodilator, increasing airflow to the lungs and helping with conditions such as asthma. Pinene is reported to be both an analgesic and anti-inflammatory. Pinene is reported to be an antioxidant and an aid in memory retention. Pinene has been reported to be useful as an analgesic, antibacterial, anti-inflammatory, anti-Proliferative, and antioxidant.

Linalool is safe with LD50 of 3 g/kg compared to that of nicotine which is 40-60 mg/kg. Linalool has a pleasant lavender scent, and has been used as a relaxant and as a treatment for anxiety. Linalool is reported useful as an analgesic, antidepressant, anti-epileptic, anti-inflammatory, anti-psychotic, anxiolytic, and sedative. Linalool has been reported to be a major anti-inflammatory agent in the essential oils that contain it.

Terpene carriers of the present invention are particularly useful for delivery of cannabinoids. Cannabinoids are a class of diverse chemical compounds that act on cannabinoid receptors on cells that repress neurotransmitter release in the brain. Ligands for these receptor proteins include the endocannabinoids (produced naturally in the body by humans and animals) [Pacher, P. et al. (2006) Pharmacological Reviews 58 (3): 389-462] the phytocannabinoids (found in cannabis and some other plants), and synthetic cannabinoids (manufactured artificially). Phytocannabinoids are of most useful in the compositions and methods of this invention. The most notable cannabinoid is the phytocannabinoid tetrahydrocannabinol (THC), the primary psychoactive compound of cannabis [Lambert, A. et al. (2005) Journal of Medicinal Chemistry 48 (16): 5059-87; Pertwee, Roger, ed. (2005). Cannabinoids. Springer-Verlag. p. 2. ISBN 3-540-22565-X.] Cannabidiol (CBD) is another major constituent of the plant. There are at least 85 different cannabinoids isolated from cannabis, exhibiting varied effects. El-Alfy, A. T. et al. (2010). Pharmacology Biochemistry and Behavior 95 (4): 434-42.] Each reference cited in the forgoing paragraph is incorporated by reference herein in its entirety for its description of cannabinoids, structures and properties thereof.

Specific cannabinoids useful in the invention are discussed hereafter. CBD is “non-psychoactive”, in that it does not produce the euphoria, time dilation, or anxiety normally induced by THC, and has been reported to be valuable in the treatment of seizure disorders such as MS and Epilepsy. Its lack of psychoactivity makes it useful in treating children, the elderly and patients that prefer to remain clear headed and focused. CBD is reported to be often as effective as THC in the management of pain and tumors, without the psychoactive effect. CBD is also reported to lower blood sugar, and to be useful in the treatment of diabetes. CBD is reported to have a calming effect, and to be useful in the treatment of stress related disorders and sleep loss. As shown herein, when combined with specific terpenes, myrcene and beta caryophyllene in particular, the typical effects produced by cannabidiol are dramatically enhanced.

Cannabichromene (CBC) is also non psychoactive, and has been reported to be about ten times more effective than CBD in the treatment of anxiety and stress. It also is reported to be useful in treating inflammation, for pain relief and to exhibit both anti-viral and anti-tumor activity. CBC has been shown to stimulate the growth of bone tissue. A combination of CBC with beta-caryophyllene and myrcene, will dramatically magnify the properties of CBC.

Another non-psychoactive, yet highly beneficial cannabinoid is cannabigerol. Cannabigerol (CBG) is non psychoactive, and has been shown to stimulate the growth of new brain cells, including in the elderly; it should be noted that genuinely neurogenic compounds are extremely rare. CBG also stimulates bone growth, is antibacterial and anti-tumor, and combats insomnia. In combination with specific terpenes, the anti-anxiety and other beneficial effects of CBG to be dramatically enhanced.

Psychoactive cannabinoids are nonselective natural ligands for cannabinoid receptor type CB1 and CB2 receptors. The CB1 receptor is understood to be associated with psychomodulatory effects. The CB2 receptor is potential associated with modulation of inflammation, pain, atherosclerosis, and osteoporosis. THC exhibits psychoactive CB1 agonist activity effects in vivo.

Preferably cannabinoids of the invention are phytocannabinoids isolated by extraction from plant sources. In specific embodiments, a cannabinoid employed is purified to contain less than 2% by weight other cannabinoids. In specific embodiments, cannabinoids may be used in the form of an extraction mixture from plant material where the extraction mixture contains from 10-99% of one or more cannabinoids. Cannabinoids can be extracted from plant material using an organic solvent as is understood in the art. Solvents such as butane, hexane, isopropyl alcohols and ethanol as well as mixtures thereof can be employed. When such extracts are employed, the extraction solvent may be removed at least in part to provide a concentrated extract. Of particular interest are extracts containing a combination of THC and CBD where the cannabinoid content in the extract ranges from 10% to 99%. It will be understood that in certain embodiments, extracts may contain relatively small amounts of terpenes which will not be detrimental to the functioning of the terpene solutions herein. Extracts containing one or more cannabinoid, particularly THC, CBD or a combination of CBD and THC can be added to terpene mixtures herein for administration by vaporization, spraying or formation of liposomes.

The invention provides terpene solutions containing CBD for reducing anxiety induced by use of THC (either taken orally (in capsule or ingested in a foodstuff, or via smoking). The invention provides terpene solutions containing CBD for reducing anxiety in general. The invention provides terpene solutions containing CBD for reduction of inflammation.

The invention is also directed to liposomal formulations for use for delivery to an individual. Liposomes are formed by any means known in the art and specially as exemplified herein by combining an amphiphilic material with a terpene mixture containing one or more active ingredients dissolved therein. Typically, the combination of amphiphilic material and terpene mixture is added to water or an aqueous solution and vigorously mixed, such as in a sonicator, to form liposomes. The ratio of amphiphilic material to terpene mixture ranges from 0.1:1 to 1.5 to 1 or from 0.8 to 1 to 1:0.8 and in specific embodiments is 0.5:1, 1:1 or 1.2 to 1. One of ordinary skill in the art can selected appropriate amphiphilic material for formation of liposome with the terpene compositions herein. Various art-known methods for forming liposomes can be employed in this invention. In specific embodiments, liposomal formulations can contain beta-caryophyllene alone or a mixture of beta-caryophyllene and myrcene. In additional embodiments, liposomal formulation can contain a mixture of beta-caryophyllene with limonene or linolool or a mixture of beta-caryophyllene and myrcene with limonene or linolool.

Nicotine can be delivered by dissolution in a terpene mixture of the invention followed by vaporization or formation of liposomes. In specific embodiments, nicotine is present in the terpene composition in an amount ranging from 0.5 to 5% by weight and more preferably in an amount ranging from 0.5 to 2.5% by weight. More specifically, nicotine is present in an amount of 2% by weight. In specific embodiments, terpene solutions containing nicotine comprise myrcene and beta-caryophyllene in approximately equal amounts by weight (weight ratio of myrcene to beta-caryophyllene ranges from 0.9:1 to 1:0.9. In specific embodiments, terpene solutions containing nicotine comprise myrcene and beta-caryophyllene in weight ratio of myrcene to beta-caryophyllene ranging from 4:1 to 2:1. In specific embodiments, terpene solutions containing nicotine comprise myrcene and beta-caryophyllene in weight ratio of myrcene to beta-caryophyllene ranging from 3.3:1 to 2.7:1. In specific embodiments, terpene solutions containing nicotine comprise myrcene and beta-caryophyllene in weight ratio of myrcene to beta-caryophyllene ranging from 3.1:1 to 2.9:1. In specific embodiments of the forgoing embodiments, myrcene can be replaced in whole or in part with neurolidol. More specifically, terpene solutions containing nicotine comprise a combination of neurolidol, myrcene and beta-caryophyllene wherein the weight ratio of neurolidol:myrcene ranges from 1:0.1 to 0.1 to 1. More specifically the weight ratio of neurolidol:myrcene ranges from 1:0.2 to 1:0.5. More specifically the weight ratio of neurolidol:myrcene ranges from 0.2:1 to 0.5 to 1. Terpene solutions containing nicotine optionally contain alpa-pinene or terpineol in the range of 0.2 to 3% or 0.2% to 2.2% by weight.

In specific embodiments, terpene solutions containing nicotine contain a terpene mixture comprising a combination of myrcene and beta-caryophyllene with minor amounts of limonene or linolool (1% to 5%) and optionally one or more of alpa-pinene, terpineol, terpinolene or menthol.

The invention is further directed to methods of delivery of one or more active ingredients to an individual using terpene mixtures herein as carriers for vaporization or carriers for spray application. Terpene compositions comprising one or more active ingredients can be delivered by any know vaporization method. For example, terpene compositions can be delivered by use of electronic heating devices, such as e-cigarette devices. Liposomal compositions of the invention can be administered by any art recognized appropriate means and specifically can be administered orally, nasally or topically.

The individual is an animal, particularly a mammal and more particularly a human.

All references throughout this application, for example patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a group of substituents is disclosed herein, it is understood that all individual members of those groups and all subgroups, including any isomers and enantiomers of the group members, and classes of compounds that can be formed using the substituents are disclosed separately. When a compound is claimed, it should be understood that compounds known in the art including the compounds disclosed in the references disclosed herein are not intended to be included. When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination.

One of ordinary skill in the art will appreciate that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

### THE EXAMPLES

### Example 1

### Example of Formulation of Terpene Mixture with CBD

To a 5 gram vial is added 300 mg beta-caryophyllene, 100 mg of myrcene, and 50 mg linalool. The vial is then capped and the vortexed for 20 seconds to fully mix the terpenes. Then 50 mg>99% white, crystalline cannabidiol (CBD) is then added and the capped vial is heated to 115° C. for 10 minutes with intermittent vortexing to fully dissolve the CBD into the terpene mixture, resulting in a clear white liquid.

### Example 2

### Example of Formulation of Mixture with Delta-9-THC

To a 5 gram vial is added 300 mg beta-caryophyllene, 100 mg of myrcene, and 50 mg linalool. The vial is then capped and the vortexed for 20 seconds to fully mix the terpenes. Then 50 mg>99% white, crystalline delta 9 tetrahydrocannabinol (THC) is then added and the capped vial is heated to 115° C. for 10 minutes with intermittent vortexing to fully dissolve the THC into the terpene mixture, resulting in a clear liquid.

### Example 3

### Example of Formulation of Mixture with Nicotine

To a 5 gram vial is added 300 mg beta-caryophyllene, 100 mg of myrcene, and 50 mg linalool. The vial is then capped and the vortexed for 20 seconds to fully mix the terpenes. Then 20 mg>99% liquid nicotine is then added by syringe and the capped vial is heated to 115° C. for 10 minutes with intermittent vortexing to fully dissolve the nicotine into the terpene mixture, resulting in a clear liquid.

### Example 4

### Example of Formulation of Liposomal Mixture Containing CBD and Terpenes Made with Lecithin as Encapsulating Agent Using Water

To 500 mg of the mixture made in part a. above is added 10 ml of distilled water, 500 mg lecithin, and blended at low speed until a homogeneous mixture is obtained. This mixture is then placed in a stainless steel ultrasonicator for 30 minutes at 35° C. resulting in a milky liposomal suspension of terpene/CBD mix in water, appropriate for use in an atomizer or other dispersal device.

### Example 5

### Example of Formulation of Liposomal Mixture Containing CBD and Terpenes Made with Lecithin as Encapsulating Agent and Organic Solvent

To 500 mg of the mixture made in part a. above is added 10 ml of chloroform 500 mg lecithin, and mixed at low speed until a homogeneous solution was obtained. The solvent was removed using a rotoevaporator under vacuum until organic solvent was removed. This mixture was then suspended in 10 ml of distilled water using a blender at low speed. This mixture was then placed in a stainless steel ultrasonicator for 30 minutes at 35° C. resulting in a clear liposomal suspension of terpene/CBD mix in water, appropriate for use in an atomizer or other dispersal device.

### Example 6

### Example of Formulation of Liposomal Mixture Containing CBD and Terpenes Made with Lecithin as Encapsulating Agent, and Ethanol to Improve the Number of Liposomes

To 1000 mg of a Terpene mixture containing 900 mg beta-Caryophyllene and 100 mg of Linalool is added 100 mg Cannabidiol. Ethanol (1 ml, 95% ethanol) is then added dropwise, followed by 600 mg of 22% lecithin. The mixture is placed in a blender at low speed, then 5 ml distilled water added dropwise and sonicated for 30 minutes at 35° C., resulting in a slightly turbid liposomal suspension suitable for application by an aerosol device such as a misting pump bottle.

### Example 7

Table 1 provides results of administration CBD by vaporization of the terpene solutions listed in the table to individuals 2 hours after administration of 30 mg of THC administered orally via capsules. The level of anxiety exhibited by the individual after administration of CBD is assessed by a psychologists on a scale ranging from −5 to +5 (full-on panic attack to extreme euphoria). The terpene solutions containing CDB were assessed with respect to s control delivery solution where the carrier was a combination of propylene glycol/glycerine. It was noted that it was difficult to maintain 10% by weight CBD in the propylene/glycol mixture. The terpene mixture could be employed to prepare CBD solution containing at least 30% by weight CBD. The terpene solutions administered exhibited significant beneficial effect lowering anxiety. The Table also includes several exemplary liposomal formulation and shows their effect on anxiety as described above.

### Example 8

Table 2 provides a summary of terpene solutions employed for delivery of nicotine by vaporization.

TABLE 1
Table of Terpene Mixtures

| | | Peak effect after | | | | | | | | |
|---|---|---|---|---|---|---|---|---|---|---|
| # of carbons | Mixture (MX) | 2 hrs for 30 mg | 60017 | 60018 | 60019 | | | | | |
| in Terpene | COMPOUNDS | of THC prior to medicating | carrier | carrier | carrier | 60020 | 60021 | 60022 | 60023 | 60024 |
| C24—(OH)2 | CBD | | 0% | 0% | 0% | 10% | 10% | 20% | 10% | 20% |
| C2—(OH)3 | PROPYLENE GLYCOL | | 50% | | | | 45% | | | |
| C3(OH)3 | GLYCERINE | | 50% | | | | 45% | | | |
| C10 | MYRCENE | | | 47% | 71% | 63% | | 57% | 63% | 57% |
| C15 | BETA CARYOPHYLLENE | | | 47% | 24% | 22% | | 19% | 22% | 19% |
| C10 | LIMONENE | | | | | | | | 5% | 4% |
| C10—OH | LINOLOOL | | | 6% | 5% | 5% | | 4% | | |
| C10 | ALPHA PINENE | | | | | | | | | |
| C10—OH | NEROLIDOL | | | | | | | | | |
| C10 | TERPINOLENE | | | | | | | | | |
| total | Total | | 100% | 100% | 100% | 100% | 100% | 100% | 100% | 100% |
| anxiety rating | −5 = full panic a [symbol] | −5 | −5 | 2 | 2 | 4 | 1 | 5 | 3 | 4 |
| (−5 to +5) | | | | | | | | | | |
| heart rate | (per/minute) | 130 | 130 | | | | | 90 | | |
| Respiratory rate | (per/minute) | 18 | 18 | | | | | 10 | | |
| (per bre [symbol] | | | | | | | | | | |
| Blood pressure (diastolic/systolic) | | 155/110 | 150/110 | | | | | 110/90 | | |
| time until peak effect (seconds) | | — | — | 120 | 120 | 30 | 120 | 30 | 30 | 30 |
| use | | | | | | vape | vape | vape | vape | vape |
| | | Peak effect after | | | | | | | | |
| # of carbons | Mixture (MX) | 2 hrs for 30 mg | | | | | 60041 | 60042 | 60043 | 60044 |
| in Terpene | COMPOUNDS | of THC prior to medicating | 60025 | 60026 | 60039 | 60040 | carrier | carrier | carrier | carrier |
| C24—(OH)2 | CBD | | 20% | 10% | 20% | 10% | 0% | 0% | 15% | 0% |
| C2—(OH)3 | PROPYLENE GLYCOL | | | | | | | | | |
| C3(OH)3 | GLYCERINE | | | | | | | | | |
| C10 | MYRCENE | | 8% | 62% | 50% | 56% | 64% | 71% | 40% | |
| C15 | BETA CARYOPHYLLENE | | 17% | 21% | 25% | 28% | 32% | 24% | 40% | 31% |
| C10 | LIMONENE | | | | | | | 5% | | |
| C10—OH | LINOLOOL | | 4% | 5% | 5% | 6% | 4% | | 5% | 7% |
| C10 | ALPHA PINENE | | | | | | | | | |
| C10—OH | NEROLIDOL | | 51% | | | | | | | 62% |
| C10 | TERPINOLENE | | | 2% | | | | | | |
| total | Total | | 100% | 100% | 100% | 100% | 100% | 100% | 100% | 100% |
| anxiety rating | −5 = full panic attack, +5 = extreme euphoria | | 5 | 4 | 5 | 4 | 2 | 2 | 2 | 2 |
| (−5 to +5) | | | | | | | | | | |
| heart rate | (per/minute) | | | | | | | | | |
| Respiratory rate | (per/minute) | | | | | | | | | |
| (per bre [symbol] | | | | | | | | | | |
| Blood pressure (diastolic/systolic) | | | | | | | | | | |
| time until peak effect (seconds) | | — | 30 | 30 | 30 | 30 | 30 | 30 | 30 | |
| use | | | vape | vape | vape | vape | vape | vape | vape | vape |
| | | Peak effect after | | | | | | | | |
| # of carbons | Mixture (MX) | 2 hrs for 30 mg | | | | 60048 | | | | 60052 |
| in Terpene | COMPOUNDS | of THC prior to medicating | 60045 | 60046 | 60047 | host | 60049 | 60050 | 60051 | host |
| C24—(OH)2 | CBD | | 10% | 0% | 10% | 0% | 10% | 5% | 2% | 0% |
| C2—(OH)3 | PROPYLENE GLYCOL | | | | | | | | | |
| C3(OH)3 | GLYCERINE | | | | | | | | | |
| C10 | MYRCENE | | | | | 33% | 28% | 29% | 30% | 57% |
| C15 | BETA CARYOPHYLLENE | | 42% | 32% | 28% | 28% | 28% | 29% | 30% | 28% |
| C10 | LIMONENE | | | | | | | | | |
| C10—OH | LINOLOOL | | 6% | 4% | 6% | 6% | 6% | 7% | 8% | 5% |
| C10 | ALPHA PINENE | | | | | | | | | |
| C10—OH | NEROLIDOL | | 42% | 64% | 56% | 33% | 28% | 29% | 30% | 10% |
| C10 | TERPINOLENE | | | | | | | | | |
| total | Total | | 100% | 100% | 100% | 100% | 100% | 100% | 100% | 100% |
| anxiety rating | −5 = full panic attack, +5 = extreme euphoria | | 4 | | 4 | 4 | 4 | 4 | 3 | 1 |
| (−5 to +5) | | | | | | | | | | |
| heart rate | (per/minute) | | | | | | | | | |
| Respiratory rate | (per/minute) | | | | | | | | | |
| (per bre [symbol] | | | | | | | | | | |
| Blood pressure (diastolic/systolic) | | | | | | | | | | |
| time until peak effect (seconds) | | — | 30 | | 30 | 30 | 30 | 30 | | |
| use | | | vape | vape | vape | vape | vape | vape | vape | vape |

| | | Peak effect after | | | |
|---|---|---|---|---|---|
| # of carbons | Mixture (MX) | 2 hrs for 30 mg | | | |
| in Terpene | COMPOUNDS | of THC prior to medicating | 60053 | 60054 | 60055 |
| C24—(OH)2 | CBD | | 10% | 10% | 10% |
| C2—(OH)3 | PROPYLENE GLYCOL | | | | |
| C3(OH)3 | GLYCERINE | | | | |
| C10 | MYRCENE | | 51% | 40% | 0% |
| C15 | BETA CARYOPHYLLENE | | 25% | 40% | 80% |
| C10 | LIMONENE | | | | |
| C10—OH | LINOLOOL | | 5% | 10% | 10% |
| C10 | ALPHA PINENE | | | | |
| C10—OH | NEROLIDOL | | 9% | | 0% |
| C10 | TERPINOLENE | | | | |
| total | Total | | 100% | 100% | 100% |
| anxiety rating | −5 = full panic attack, +5 = extreme euphoria | | 4 | 4 | 4 |
| (−5 to +5) | | | | | |
| heart rate | (per/minute) | | | | |
| Respiratory rate | (per/minute) | | | | |
| (per bre [symbol] | | | | | |
| Blood pressure (diastolic/systolic) | | | | | |
| time until peak effect (seconds) | | — | | | |
| use | | | vape | Liposomal | Liposomal |

[symbol]

indicates data missing or illegible when filed

TABLE 2
EXEMPLARY MIXTURES CONTAINING NICOTINE

| | Mixture (MX) | 60017 | 60028 | 60029 | | | | | | | | | |
|---|---|---|---|---|---|---|---|---|---|---|---|---|---|
| Terpene | COMPOUNDS | carrier | carrier | carrier | 60030 | 60031 | 60032 | 60033 | 60034 | 60035 | 60036 | 60037 | 60038 |
| | | Weight % | | | | | | | | | | | |
| | NICOTINE | 0% | 0% | 0% | 2% | 2% | 2% | 2% | 2% | 2% | 2% | 2% | 2% |
| | PROPYLENE | 50% | | | | | 49% | | | | | | |
| | GLYCOL | | | | | | | | | | | | |
| | GLYCERINE | 50% | | | | | 49% | | | | | | |
| C10 | MYRCENE | | 47% | 71% | 71% | 71% | | 70% | 69% | 69% | 16% | 68% | 69% |
| C15 | BETA | | 47% | 24% | 24% | 24% | | 24% | 24% | 25% | 23% | 23% | 24% |
| | CARYOPHYLLENE | | | | | | | | | | | | |
| C10 | LIMONENE | | | | 5% | | | | 5% | 4% | 4% | | |
| C10 | LINALOOL | | | 5% | | 5% | | 4% | | | 4% | 5% | |
| C10 | ALPHA PINENE | | | | | | | | | | | | |
| C10 | NEROLIDOL | | | | | | | | | | 51% | | |
| C10 | TERPINOLENE | | | | | | | | | | | 2% | |
| C15 | Menthol | | 6% | | | | | | | | | | 5% |
| total | | 100% | 100% | 100% | 100% | 100% | 100% | 100% | 100% | 100% | 100% | 100% | 100% |

1. A terpene carrier composition comprising a mixture of terpenes as a carrier for the one or more active ingredient for vaporizers or liposomal transport wherein the mixture of terpenes comprises a first terpene selected from myrcene, nerolidol, beta caryophyllene or a mixture thereof and one or more second terpenes selected from the group consisting of limonene, pinene, linalool, terpineol, terpinolene, terpineol, menthol and a mixture thereof.

2. The composition of claim 1 comprising a mixture of two or more terpenes selected from the group consisting of myrcene, beta-caryophyllene, linalool, or limonene.

3. The composition of claim 1 comprising a mixture of myrcene and beta-caryophyllene and one of linalool or limonene.

4. The composition of claim 1 comprising a mixture of beta-caryophyllene, one of linalool or limonene and a combination of myrcene and neurolidol.

5. The composition of claim 1, wherein the amount of first terpene present ranges from 50% to 100% by weight.

6. The composition of claim 1, wherein the amount of second terpene present ranges from 1% to 25% by weight.

7. The composition of claim 1, wherein the first terpene is a mixture of myrcene and beta-caryophyllene, where the weight ratio of myrcene to beta-caryophyllene ranges from 4:1 to 1:4.

8. The composition of claim 7, wherein the weight ratio of myrcene to beta-caryophyllene is 1:1.

9. A composition of claim 1 further comprising one or more isolated cannabinoid receptor modulator or nicotine.

10. The composition of claim 9, wherein the composition comprises 10% by weight or more of the one or more isolated cannabinoid receptor modulators.

11. The composition of claim 10, wherein the one or more isolated cannabinoid receptor modulator is selected from THC, CBD, CBC, CBG or a mixture thereof.

12. The composition of claim 11, wherein the one or more isolated cannabinoid receptor modulator is selected from THC or a mixture of THC with CBD or CDG.

13. A composition comprising cannabidiol and a mixture of terpenes of claim 1 and an amphiphilic material to form liposomes as a carrier for liposomal delivery.

14. The composition of claim 13, wherein the amphiphillic material is lecithin or phosphatityl choline.

15. A method for delivery of one or more active ingredient comprising the steps of dissolving the one or more active ingredients in a mixture of terpenes of claim 1 to form a delivery composition and vaporizing the vaporizing composition.

16. The method of claim 15, wherein the one or more active ingredient is one or more isolated cannabinoid receptor modulator.

17. The method of claim 16, wherein the one or more active ingredient is THC or a mixture of THC with CBD or CDG.

18. The method of claim 15, wherein the one or more active ingredient is nicotine.

19. A method for delivery of one or more active ingredient comprising the steps of dissolving the one or more active ingredients in a mixture of terpenes of claim 1 to form a delivery composition and spraying the delivery composition in contact with skin or mucosal tissue for delivery.

20. A method for delivery of one or more active ingredients to an individual comprising the steps of dissolving the one or more active ingredients in a mixture of terpenes of claim 1 to form a delivery composition, forming a liposomal composition comprising the delivery composition and administering the liposomal composition to the individual.
